(19) United States
(12) Patent Application Publication
(10) Pub. No.: US 2026/0175251 A1
(43) Pub. Date: Jun. 25, 2026

(54) DEVICE FOR DIFFUSING VOLATILE SUBSTANCES

(71) Applicant: ZOBELE HOLDING SPA, Trento (IT)

(72) Inventors: Sergio Luque Vera, Barcelona (ES); José María García Jiménez, Barcelona (ES); Li Zhiheng, Barcelona (ES)

(73) Assignee: ZOBELE HOLDING SPA, Trento (IT)

(21) Appl. No.: 19/128,951

(22) PCT Filed: Nov. 10, 2023
(86) PCT No.: PCT/EP2023/081460

§ 371 Date: May 9, 2025

(30) Foreign Application Priority Data
Nov. 10, 2022 (ES) P202230969

(51) Int. Cl. B05B 17/00; A61L 9/14; B05B 17/06

(52) U.S. Cl. CPC B05B 17/0646; A61L 9/14; B05B 17/0684; A61L 2209/111; A61L 2209/132; A61L 2209/133

(57) ABSTRACT

Described is a device for diffusing volatile substances. The device includes a container for holding a liquid containing the volatile substances, a piezoelectric element, the vibration of which causes the diffusion of the volatile substances, and a control electronics that applies an electrical signal to the piezoelectric element, adjusting it to operate at its resonance frequency. The control electronics searches for the resonance frequency and analyses the electrical signal to determine whether or not the liquid is present in the container based on that resonance frequency.

### OBJECT OF THE INVENTION

The present invention relates to a device for diffusing volatile substances, which makes it possible to detect the end of useful life of a liquid containing the volatile substances.

### BACKGROUND TO THE INVENTION

Electrical devices, powered by batteries, are known for diffusing volatile substances. Electric diffusers are devices for diffusing volatile substances which use electrical energy to activate heating elements, diffuser elements or dispensing elements. These heating elements are intended to optimise efficiency, dose control and user control.

This type of device is designed to be used without the need for air currents or temperature restrictions, while still achieving good evaporation and/or diffusion of volatile substances.

Some of these devices include motors, heaters or solenoid valves that allow vapours to escape through nozzles or wicks. These devices are based on a container for the volatile substances, an electrical system to allow diffusion and an energy system to power the system.

Among all the devices for diffusing volatile substances, one stands out above the rest, the main advantage of which is fogging.

These are electronic devices comprising a piezoelectric element that is in contact with the liquid containing the volatile substances, either directly or through a capillary element.

The piezoelectric element vibrates when supplied with an alternating voltage at a resonant frequency specified by the piezoelectric element manufacturer. This vibration causes misting to occur.

One of the problems with this type of system is the end-of-life detection of volatile substances in the device.

Alternative solutions for detecting the end of life of a liquid refill are known, as in diffusion devices that work by nebulising a container with liquid it is interesting to alert the user of the end of life of the liquid in order to anticipate its replacement.

One solution for the end-of-life indication of a liquid is to define an opening in the container in order to verify the liquid level. In some devices it is not so obvious due to the lighting of the space, the location of the device in the working area or elements that make it physically difficult to verify the end of life of the liquid, usually being the olfactory sense the mechanism to detect the total consumption.

There are solutions that translate the end-of-life of a deposited liquid into an electrical signal which in turn is transformed into a light or audible signal to attract the user's attention. These solutions are based on measuring the change in the physical properties of the liquid, e.g. using light waves.

When light waves are used for liquid level detection, systems are known to detect the liquid at the base of a container or in a wick.

Detection systems that are positioned at the base of the container have the following disadvantages:
Difficulty of reproducibility due to the lack of geometric accuracy in the generally used refills.
Influence of external elements, such as sunlight or reflections.
The end of life is detected when there is still liquid in the wick and there is still diffusion of substance.

Detection systems that are positioned inside the fuse have the following disadvantages:
There is a requirement to use transparent wicks, so that, in the presence of liquid inside the wick, a sufficient amount of light can refract through the wick cavities and be received by a sensor.
The position of the light emitter must be adjusted according to the refractive index of the formulation to be measured.
There is influence from external agents such as sunlight or reflections.

Some end-of-life indicators using light have a light emitter and a receiver around the container in misaligned positions. When there is liquid in the container and sensors are calibrated to the refraction of the liquid, the operation is correct, but when there is no liquid, the light travels in a straight line and does not reach the receiver, so that repeatability of different liquids with different refractive indices is prevented.

There are detection systems by measuring the electrical conductivity in the liquid by flowing an electric current through the liquid providing a conductivity value of the liquid. These systems have the following disadvantages:
Requires the presence of a measuring element and its calibration.
It involves additional energy consumption.
If it is not properly calibrated and covers the entire container, it may give incorrect values.

When sensors measuring dielectric capacitance are used, their value is a function of the distance to the material to be measured, and has similar disadvantages to electrical conductivity systems.

Sensors can also be used to measure the weight of the liquid inside the container, which provides force data that, measured with a strain gauge, can be translated into an electrical signal and identify an end of life of the liquid by weight variation.

In common with the previous solutions, an additional component, i.e. a sensor, is required to obtain the information.

Changing the type of refill and thus its shape, weight, density and quantity of liquid causes the measurement reference to be lost and an identification system is additionally required.

### DESCRIPTION OF THE INVENTION

Therefore, an objective of the present invention is to provide a device for diffusing volatile substances, which allows to detect the end-of-life of a refill with liquid, by using a piezoelectric element, without employing additional components, such as sensors.

With the device for diffusing volatile substances of the invention, the aforementioned disadvantages are solved, presenting other advantages that will be described below.

The volatile substance diffusion device according to the present invention is described in claim 1, and the dependent claims include additional features that are optional.

In particular, the device for diffusing volatile substances according to the present invention comprises:
a container for holding a liquid containing the volatile substances;
a piezoelectric element, the vibration of which causes the diffusion of volatile substances; and
a control electronics that applies an electrical signal to the piezoelectric element, adjusting it to operate at its resonant frequency,
in which the control electronics searches for the resonance frequency and analyses the electrical signal to determine whether or not liquid is present in the container based on that resonance frequency.

Preferably, the control electronics determine whether or not there is liquid in the container based on the drop in consumption during the search for the resonant frequency.

In particular, the drop in consumption during the search for the resonant frequency is determined by an angle of the current signal, such that if the angle is greater than a predetermined value, it is determined that there is liquid in the container, and if the angle is less than a predetermined value, it is determined that there is no liquid in the container.

According to an embodiment, the device for diffusing volatile substances according to the present invention also comprises a capillary element partially housed in said container.

Furthermore, the device for diffusing volatile substances according to the present invention also comprises a perforated plate through which the volatile substances are diffused, the perforated plate being in contact with the piezoelectric element, the perforated plate preferably being in contact with the capillary element.

According to another embodiment, the piezoelectric element is in contact with the liquid. The device for diffusion of volatile substances according to the present invention provides the following advantages, among others:
The volatile substance diffusion device according to the present invention allows to identify a variation in the physical and electrical operating properties of the piezoelectric element, and does not require additional hardware components such as probes or sensor circuits to the original device architecture.

In addition, the same electronics used in the activation of the piezoelectric element membrane are used to obtain the information.

It is a cost-saving solution, as it eliminates external agents from the piezoelectric element assembly and replacement that could influence end-of-life detention.

### BRIEF DESCRIPTION OF THE DRAWINGS

For a better understanding of what has been explained above, some drawings are included in which, schematically and only by way of a non-limiting example, a practical case of embodiment is shown.

FIG. 1 is an elevation view of a device for diffusing volatile substances according to the present invention, according to a first embodiment;

FIG. 2 is an elevation view of a device for diffusing volatile substances according to present invention, according to a second embodiment;

FIG. 3 is a graph showing the intensity as a function of frequency during the search for the resonant frequency that is generated by the piezoelectric element when the container contains liquid; and

FIG. 4 is a graph showing the intensity as a function of frequency during the search for the resonant frequency that is generated by the piezoelectric element when the container is free of liquid.

### DESCRIPTION OF A PREFERRED EMBODIMENT

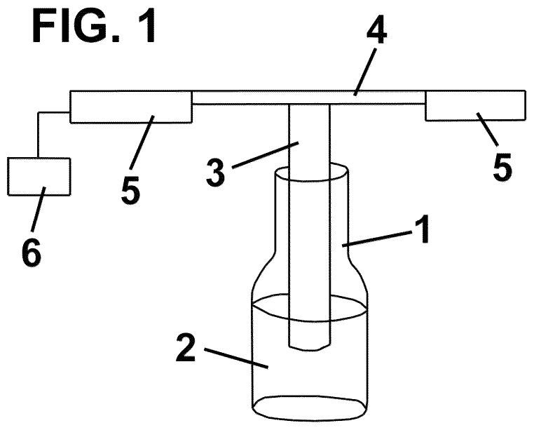

FIG. 1 shows a first embodiment of the volatile substance diffusion device according to the present invention, comprising a container 1, or refill, which houses a liquid 2 containing the volatile substances to be diffused into the environment.

The device also comprises a capillary element 3, such as a wick, which is partially immersed in the liquid 2 housed in the container 1, and a perforated plate 4, preferably made of metal.

This perforated plate 4 is in contact with a piezoelectric element 5, which receives an electrical signal from a control electronics 6, so that the vibration of the piezoelectric element 5 from this vibration causes the diffusion of the volatile substances contained in the liquid 2.

The control electronics 6 also monitors and analyses the variation of consumption during the search for the resonance frequency of the piezoelectric element 5 to detect whether or not there is liquid inside the container 1, as the consumption varies, as will be described below.

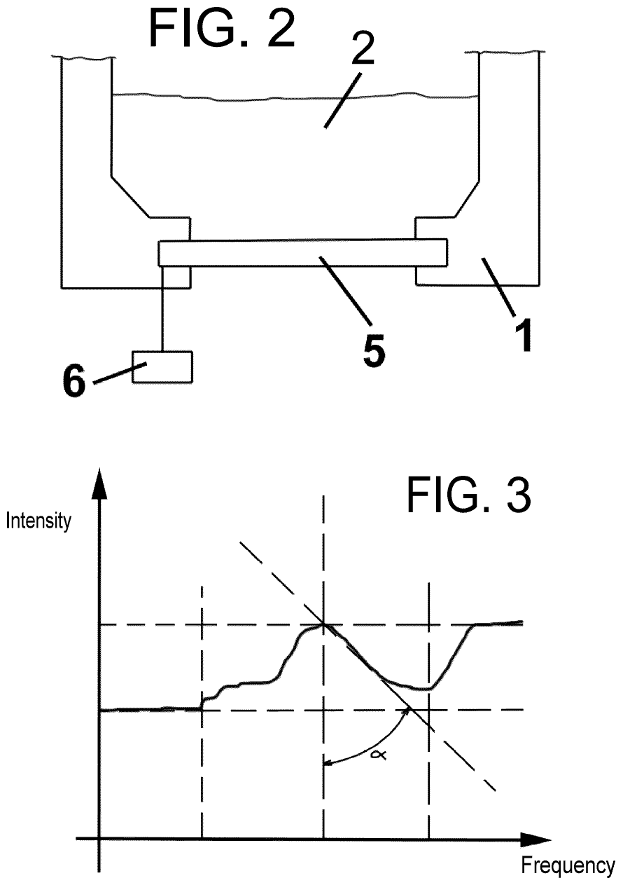

FIG. 2 shows a second embodiment of the volatile substance diffusion device according to the present invention. For the sake of simplicity, the same reference numbers are used to identify the same or similar elements as in the first embodiment. In this second embodiment, the piezoelectric element 5 is directly in contact with the liquid 2, and said piezoelectric element 5 is mounted or mechanically fixed to the container 1. In other words, unlike the first embodiment, in this second embodiment the presence of the capillary element 3 and the perforated plate 4 is not necessary.

Also in this second embodiment, the vibration of the piezoelectric element 5 causes the diffusion of the volatile substances contained in the liquid 2.

In this way, in the device for diffusing volatile substances according to the present invention it is possible to detect the end of life of the liquid substance in contact with the piezoelectric element 5 and thus inside the container 1, as described below.

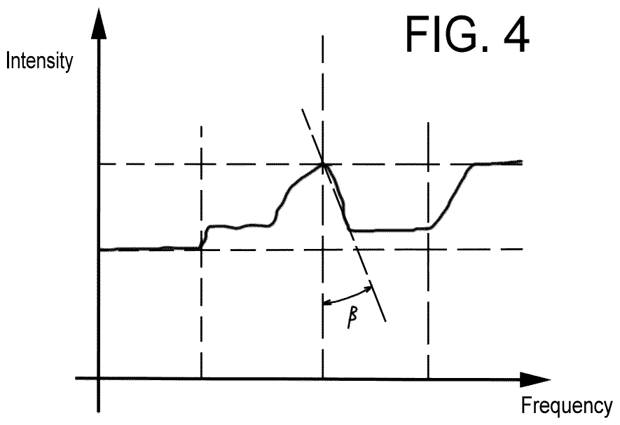

The piezoelectric element 5 is supplied with a sinusoidal voltage at a given frequency. The manufacturer of the piezoelectric element 5 sets frequency ranges, but each piezoelectric element 5 has its resonant frequency. This resonance frequency usually occurs when the piezo element 5 has a higher power consumption. The control electronics 6 is in charge of measuring this consumption at different frequencies to determine the resonance frequency at which the piezoelectric element 5 should operate. The output is a graphical representation as a function of the alteration of the intensity with respect to the frequency of the piezoelectric element 5, as shown in FIGS. 3 and 4. This representation presents a pattern that allows the identification of the absence of liquid when the representation meets defined elements of variation of the intensity curve. This pattern can be translated into an electrical signal indicating the end of life.

In particular, the control electronics 6 detects this resonance frequency and determines whether or not there is liquid 2 in the container 1 on the basis of the variation in consumption during the search for the resonance frequency, in particular on the basis of the drop in consumption during the search for the resonance frequency.

As shown in FIGS. 3 and 4, the consumption drop during the search for the resonant frequency is determined by an angle (α, β) of the current signal, so that if the angle (α) is greater than a predetermined value, FIG. 3, it is determined that there is liquid 2 in container 1, and if the angle (β) is less than a predetermined value, i.e. tends to zero, FIG. 4, it is determined that there is no liquid 2 in container 1.

When it is determined that there is no liquid 2 in the container 1, the user can be informed in any suitable manner, e.g., by a visual or light signal.

Although reference has been made to a specific embodiment of the invention, it is obvious to a person skilled in the art that the device for diffusing volatile substances described is susceptible to numerous variations and modifications, and that all the details mentioned can be replaced by technically equivalent ones, without departing from the scope of protection defined by the appended claims.

1. A device for diffusing volatile substances, comprising:
a container for holding a liquid containing the volatile substances;
a piezoelectric element, thea vibration of which causes the diffusion of the volatile substances; and
a control electronics that applies an electrical signal to the piezoelectric element, adjusting it to operate at its resonant frequency, characterized in that
the control electronics searches for the resonant frequency and analyses the electrical signal to determine whether or not the liquid is present in the container based on the resonant frequency.

2. The device for diffusing volatile substances according to claim 1, wherein the control electronics determines whether or not the liquid is present in the container on the basis of thea consumption drop after the resonance frequency.

3. The device for diffusing volatile substances according to claim 2, wherein the consumption drop after the resonance frequency is determined by an angle (α, β) of thea current signal, such that if the angle (α) is greater than a predetermined value, it is determined that the liquid is in the container, and if the angle (β) is less than a predetermined value, it is determined that the liquid is not in the container.

4. The device for diffusing volatile substances according to claim 1, comprising a capillary element partially housed in the container.

5. The device for diffusing volatile substances according to claim 4 comprising a perforated plate through which the volatile substances are diffused, the perforated plate being in contact with the piezoelectric element.

6. The device for diffusing volatile substances according to claim 5, wherein the perforated plate is in contact with the capillary element.

7. The device for diffusing volatile substances according to claim 1, wherein the piezoelectric element is in contact with the liquid.
